(19) United States
(12) Patent Application Publication
(10) Pub. No.: US 2023/0148991 A1
(43) Pub. Date: May 18, 2023

(54) AUTOMATICALLY DETECTING AND QUANTIFYING ANATOMICAL STRUCTURES IN AN ULTRASOUND IMAGE USING A CUSTOMIZED SHAPE PRIOR

(71) Applicant: EchoNous, Inc., Redmond, WA (US)

(72) Inventors: Fan Zhang, Bellevue, WA (US); Babajide Ayinde, Redmond, WA (US)

(21) Appl. No.: 17/529,565

(22) Filed: Nov. 18, 2021

(51) Int. Cl. A61B 8/08; G06K 9/00; G06K 9/32; G16H 50/20; G16H 30/20; A61B 8/00; A61B 8/06

(52) U.S. Cl. CPC A61B 8/0891; G06K 9/00979; G06K 9/3241; G06K 9/3208; G16H 50/20; G16H 30/20; A61B 8/488; A61B 8/461; A61B 8/06; A61B 8/0883

(57) ABSTRACT

A facility for detecting a target structure is described. The facility receives an ultrasound image. It subjects the ultrasound image to a detection model to obtain, for each of one or more occurrences of a target structure appearing in the ultrasound image, a set of parameter values fitting a distinguished shape to the target structure occurrence. The facility stores the obtained one or more parameter value sets in connection with the ultrasound image.

### BACKGROUND

Ultrasound imaging is a useful medical imaging modality. For example, internal structures of a patient's body may be imaged before, during or after a therapeutic intervention. Also, qualitative and quantitative observations in an ultrasound image can be a basis for diagnosis. For example, ventricular volume determined via ultrasound is a basis for diagnosing, for example, ventricular systolic dysfunction and diastolic heart failure.

A healthcare professional typically holds a portable ultrasound probe, sometimes called a “transducer,” in proximity to the patient and moves the transducer as appropriate to visualize one or more target structures in a region of interest in the patient. A transducer may be placed on the surface of the body or, in some procedures, a transducer is inserted inside the patient's body. The healthcare professional coordinates the movement of the transducer so as to obtain a desired representation on a screen, such as a two-dimensional cross-section of a three-dimensional volume.

Particular views of an organ or other tissue or body feature (such as fluids, bones, joints or the like) can be clinically significant. Such views may be prescribed by clinical standards as views that should be captured by the ultrasound operator, depending on the target organ, diagnostic purpose or the like.

In some ultrasound images, it is useful to identify anatomical structures visualized in the image. For example in an ultrasound image view showing a particular organ, it can be useful to identify constituent structures within the organ. As one example, in some views of the heart, constituent structures are visible, such as the left and right atria; left and right ventricles; and aortic, mitral, pulmonary, and tricuspid valves.

Existing software solutions have sought to identify such structures automatically. These existing solutions seek to “detect” a structure by specifying a horizontal bounding box in which the structure is visible, or “segment” the structure by identifying the individual pixels in the image that show the structure.

### BRIEF DESCRIPTION OF THE DRAWINGS

FIG. 1 is a schematic illustration of a physiological sensing device, in accordance with one or more embodiments of the present disclosure.

FIG. 2 is a block diagram showing some of the components typically incorporated in at least some of the computer systems and other devices on which the facility operates.

FIG. 3 is a flow diagram showing a process performed by the facility in some embodiments to establish and train a model for identifying a particular target anatomical structure.

FIG. 4 is a flow diagram showing a process performed by the facility in some embodiments to detect instances of a target structure visualized in an ultrasound image.

FIG. 5 is a reference region layout diagram showing a grid of rectangular reference regions used by the facility in some embodiments.

FIG. 6 is a model architecture diagram showing a model architecture used by facility in some embodiments to accommodate a grid of rectangle reference regions.

FIG. 7 is an ultrasound drawing of an ultrasound image showing an aortal cross-section detected by the facility.

FIG. 8 relates to detecting LVOTs in ultrasound images of the heart. In some embodiments, the facility selects a rectangle as the shape prior to use for this application.

FIG. 9 is an ultrasound drawing of an ultrasound image showing a blood vessel interior detected by the facility.

FIG. 10 is a conceptual diagram showing aspects of the facility's estimation of blood velocity in the direction of the blood vessel's length.

FIG. 11 is a reference region layout diagram showing a series of top-to-bottom circle-sector reference regions used by the facility in some embodiments.

FIG. 12 is a model architecture diagram showing a model architecture used by facility in some embodiments to accommodate a series of circle-sector reference regions.

FIG. 13 is a first ultrasound drawing of ultrasound images showing B-lines detected by the facility.

FIG. 14 is a second ultrasound drawing of ultrasound images showing B-lines detected by the facility.

FIG. 15 is a third ultrasound drawing of ultrasound images showing B-lines detected by the facility.

### DETAILED DESCRIPTION

The inventors have recognized that conventional approaches to anatomical structure identification in ultrasound images have significant disadvantages.

In particular, the inventors have recognized that conventional detection techniques provide inadequate detail for many typical diagnostic uses of ultrasound images. As examples: (1) having a horizontal bounding box surrounding an aorta cross-section is not adequate to determine a long-axis diameter of the aorta cross-section in any orientation of the aorta cross-section to the ultrasound image; (2) having a horizontal bounding box surrounding a left ventricle outflow track (“LVOT”) is not adequate to place a Doppler sample gate to capture a pulse wave Doppler signal specific to the LVOT in any orientation of the LVOT to the ultrasound image; and (3) having a horizontal bounding box surrounding a B-line reverberation artifact in lung ultrasound is not adequate to determine the angular width of the B-line.

The inventors have further recognized that conventional segmentation techniques have a high computational resource cost, which necessitates the use of expensive, powerful computing hardware, and/or precludes real-time or near-real-time operation. Additionally, for many purposes conventional segmentation techniques are overkill, in the sense that the high level of detail of the individual-pixel surfaces that they delineate are unnecessary—and often even disadvantageous—for typical diagnostic uses of ultrasound images such as those listed above.

In response to recognizing these disadvantages, the inventors have conceived and reduced to practice a software and/or hardware facility that automatically detects and quantifies anatomical structures in an ultrasound image using a customized shape prior (“the facility”). For particular ultrasound application, the facility defines an anatomical structure whose instances are to be identified in ultrasound images, as well as a shape to fit to identified structure instances (the “shape prior”), and attributes of that shape. The facility uses this information to define and train a structure identification model for this structure. The facility applies this model to ultrasound images; for each instance of the structure visualized in the image, the model returns the attributes of the shape fitted to that structure instance. In some embodiments, the facility uses the shape's attributes to superimpose the shape in a display of the ultrasound image.

In some embodiments, the facility uses one or more of the shape's attributes as a diagnostic value for the patient. In various embodiments, the facility uses the shape's attributes for a variety of other purposes.

In some embodiments, the structure identification model used by the facility is derived from You Only Look Once (“YOLO”) models, described in (1) Redmon, Joseph, Santosh Divvala, Ross Girshick, and Ali Farhadi. “You only look once: Unified, real-time object detection,” Proceedings of the IEEE conference on computer vision and pattern recognition, pp. 779-788, 2016, available at arxiv.org/pdf/1506.02640v5.pdf; (2) Redmon, Joseph and Ali Farhadi., “YOLO9000: Better, Faster, Stronger,” University of Washington, Allen Institute for AI, 2016, available at arxiv.org/pdf/1612.08242v1.pdf; (3) Redmon, Joseph and Ali Farhadi, YOLOv3: An Incremental Improvement, University of Washington, available at pkeddie.com/media/files/papers/YOLOv3.pdf; and (4) Hurtik, Petr, et al. “Poly-YOLO: higher speed, more precise detection and instance segmentation for YOLOv3,” arXiv preprint arXiv:2005.13243, 2020, available at arxiv.org/pdf/2005.13243, each of which are hereby incorporated by reference in their entirety. In cases where a document incorporated herein by reference conflicts with the present disclosure, the present disclosure controls. Briefly, YOLO models divide each input image into rectangular regions rotationally aligned with the borders of the image. For each region, the model outputs a probability that the region contains at least part of a target structure instance, as well as attributes defining a horizontal bounding box around the structure instance that occurs in the region.

In defining its per-application structure identification models, the facility defines (a) the shape prior and its attributes, and (b) a shape, size, and arrangement of regions well-suited to the shape prior and common distributions of the shape prior in typical ultrasound images. The facility chooses an architecture for the model suited to an output in which one or more dimensions enumerate the regions, and a final dimension contains, for each region: (1) the probability that the region contains at least part of a structure instance, and (2) the attributes of the shape prior. The facility uses ultrasound images showing the structure to train the model defined as described above. Once trained, the facility applies the model to detect instances of the structure and produce the attributes of the shape prior fitted to each structure instance. In some embodiments, the facility uses the results of applying the model as a basis for instructing the operator to reposition and/or reorient the transducer, and/or adjusting Doppler analysis results based on a rotational angle of the fitted shape prior.

As one example, in some embodiments, to detect aorta cross-sections in ultrasound images of the heart, the facility defines an elliptical shape prior, and the following attributes for it: probability of presence, center X and Y coordinates, long axis diameter and rotational angle, and short axis diameter. Based upon the elliptical shape prior and common distributions of aortic cross-sections in typical ultrasound images, the facility selects a grid of rectangular regions.

As another example, in some embodiments, to detect LVOTs in ultrasound images of the heart, the facility defines a rotatable rectangle shape prior, and the following attributes for it: probability of presence, center X and Y coordinates, long axis diameter and rotational angle, and short axis diameter. Based upon the rotatable rectangle shape prior and common distributions of LVOTs in typical ultrasound images, the facility selects a grid of rectangular regions.

As an additional example, in some embodiments, to detect the interior of blood vessels in ultrasound images of them, the facility defines a rotatable rectangle shape prior, and the following attributes for it: probability of presence, center X and Y coordinates, long axis diameter and rotational angle, and short axis diameter. Based upon the rotatable rectangle shape prior and common distributions of blood vessels in typical ultrasound images, the facility selects a grid of rectangular regions.

As a further example, in some embodiments, to detect B-lines in ultrasound images of the lung, the facility defines a circle-sector shape prior defined with respect to the top and bottom of the ultrasound image cone, and the following attributes for it: probability of presence, directed angle between a line perpendicular to the center of the active surface of the probe (sometimes called the “scanning axis”) and the center of the sector, and angular width of the sector. Based upon the sector shape prior and common distributions of B-lines in typical ultrasound images, the facility selects a series of circle-sectors spanning the bottom of the ultrasound image cone. The four examples are discussed in further detail below.

By performing in some or all of these ways, the facility rapidly and efficiently identifies instances of a target anatomical structure visualized in an ultrasound image, and directly provides diagnostically-useful values for those structure instances.

Additionally, the facility improves the functioning of computer or other hardware, such as by reducing the dynamic display area, processing, storage, and/or data transmission resources needed to perform a certain task, thereby enabling the task to be permitted by less capable, capacious, and/or expensive hardware devices, and/or be performed with lesser latency, and/or preserving more of the conserved resources for use in performing other tasks. For example, by not seeking to identify every individual pixel showing a structure instance, the facility can reduce the processing load on the computing device I which the facility is implemented, permitting it to be outfitted with a less powerful and less expensive processor, or permitting it to undertake more or larger simultaneous processing tasks.

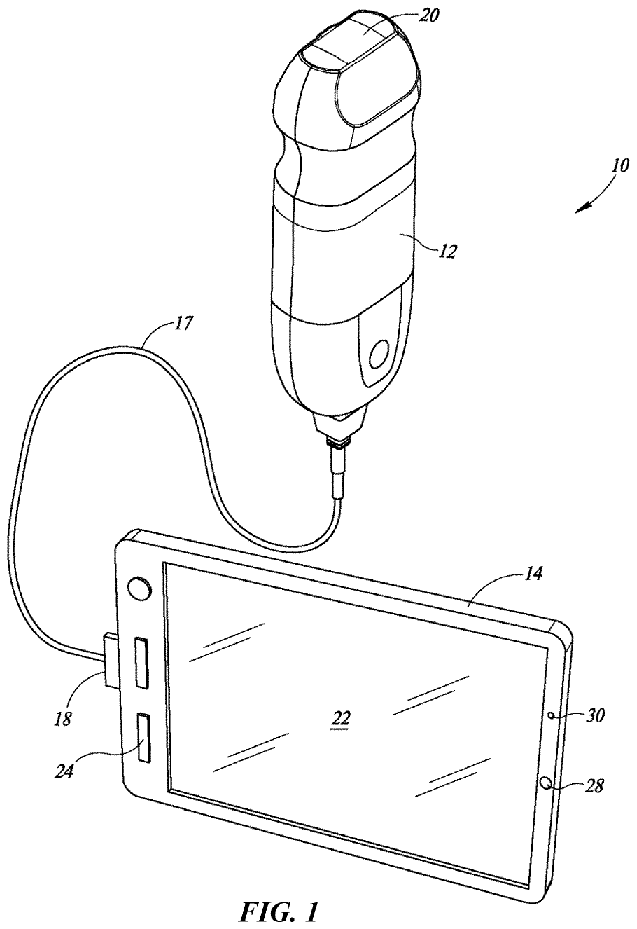

FIG. 1 is a schematic illustration of a physiological sensing device, in accordance with one or more embodiments of the present disclosure. The device 10 includes a probe 12 that, in the illustrated embodiment, is electrically coupled to a handheld computing device 14 by a cable 17. The cable 17 includes a connector 18 that detachably connects the probe 12 to the computing device 14. The handheld computing device 14 may be any portable computing device having a display, such as a tablet computer, a smartphone, or the like. In some embodiments, the probe 12 need not be electrically coupled to the handheld computing device 14, but may operate independently of the handheld computing device 14, and the probe 12 may communicate with the handheld computing device 14 via a wireless communication channel.

The probe 12 is configured to transmit an ultrasound signal toward a target structure and to receive echo signals returning from the target structure in response to transmission of the ultrasound signal. The probe 12 includes an ultrasound sensor 20 that, in various embodiments, may include an array of transducer elements (e.g., a transducer array) capable of transmitting an ultrasound signal and receiving subsequent echo signals.

The device 10 further includes processing circuitry and driving circuitry. In part, the processing circuitry controls the transmission of the ultrasound signal from the ultrasound sensor 20. The driving circuitry is operatively coupled to the ultrasound sensor 20 for driving the transmission of the ultrasound signal, e.g., in response to a control signal received from the processing circuitry. The driving circuitry and processor circuitry may be included in one or both of the probe 12 and the handheld computing device 14. The device 10 also includes a power supply that provides power to the driving circuitry for transmission of the ultrasound signal, for example, in a pulsed wave or a continuous wave mode of operation.

The ultrasound sensor 20 of the probe 12 may include one or more transmit transducer elements that transmit the ultrasound signal and one or more receive transducer elements that receive echo signals returning from a target structure in response to transmission of the ultrasound signal. In some embodiments, some or all of the transducer elements of the ultrasound sensor 20 may act as transmit transducer elements during a first period of time and as receive transducer elements during a second period of time that is different than the first period of time (i.e., the same transducer elements may be usable to transmit the ultrasound signal and to receive echo signals at different times).

The computing device 14 shown in FIG. 1 includes a display screen 22 and a user interface 24. The display screen 22 may be a display incorporating any type of display technology including, but not limited to, LCD or LED display technology. The display screen 22 is used to display one or more images generated from echo data obtained from the echo signals received in response to transmission of an ultrasound signal, and in some embodiments, the display screen 22 may be used to display color flow image information, for example, as may be provided in a Color Doppler imaging (CDI) mode. Moreover, in some embodiments, the display screen 22 may be used to display audio waveforms, such as waveforms representative of an acquired or conditioned auscultation signal.

In some embodiments, the display screen 22 may be a touch screen capable of receiving input from an operator that touches the screen. In such embodiments, the user interface 24 may include a portion or the entire display screen 22, which is capable of receiving operator input via touch. In some embodiments, the user interface 24 may include one or more buttons, knobs, switches, and the like, capable of receiving input from an operator of the ultrasound device 10. In some embodiments, the user interface 24 may include a microphone 30 capable of receiving audible input, such as voice commands.

The computing device 14 may further include one or more audio speakers 28 that may be used to output acquired or conditioned auscultation signals, or audible representations of echo signals, blood flow during Doppler ultrasound imaging, or other features derived from operation of the device 10.

The probe 12 includes a housing, which forms an external portion of the probe 12. The housing includes a sensor portion located near a distal end of the housing, and a handle portion located between a proximal end and the distal end of the housing. The handle portion is proximally located with respect to the sensor portion.

The handle portion is a portion of the housing that is gripped by an operator to hold, control, and manipulate the probe 12 during use. The handle portion may include gripping features, such as one or more detents, and in some embodiments, the handle portion may have a same general shape as portions of the housing that are distal to, or proximal to, the handle portion.

The housing surrounds internal electronic components and/or circuitry of the probe 12, including, for example, electronics such as driving circuitry, processing circuitry, oscillators, beamforming circuitry, filtering circuitry, and the like. The housing may be formed to surround or at least partially surround externally located portions of the probe 12, such as a sensing surface. The housing may be a sealed housing, such that moisture, liquid or other fluids are prevented from entering the housing. The housing may be formed of any suitable materials, and in some embodiments, the housing is formed of a plastic material. The housing may be formed of a single piece (e.g., a single material that is molded surrounding the internal components) or may be formed of two or more pieces (e.g., upper and lower halves) which are bonded or otherwise attached to one another.

In some embodiments, the probe 12 includes a motion sensor. The motion sensor is operable to sense a motion of the probe 12. The motion sensor is included in or on the probe 12 and may include, for example, one or more accelerometers, magnetometers, or gyroscopes for sensing motion of the probe 12. For example, the motion sensor may be or include any of a piezoelectric, piezoresistive, or capacitive accelerometer capable of sensing motion of the probe 12. In some embodiments, the motion sensor is a tri-axial motion sensor capable of sensing motion about any of three axes. In some embodiments, more than one motion sensor 16 is included in or on the probe 12. In some embodiments, the motion sensor includes at least one accelerometer and at least one gyroscope.

The motion sensor may be housed at least partially within the housing of the probe 12. In some embodiments, the motion sensor is positioned at or near the sensing surface of the probe 12. In some embodiments, the sensing surface is a surface which is operably brought into contact with a patient during an examination, such as for ultrasound imaging or auscultation sensing. The ultrasound sensor 20 and one or more auscultation sensors are positioned on, at, or near the sensing surface.

In some embodiments, the transducer array of the ultrasound sensor 20 is a one-dimensional (1D) array or a two-dimensional (2D) array of transducer elements. The transducer array may include piezoelectric ceramics, such as lead zirconate titanate (PZT), or may be based on microelectromechanical systems (MEMS). For example, in various embodiments, the ultrasound sensor 20 may include piezoelectric micromachined ultrasonic transducers (PMUT), which are microelectromechanical systems (MEMS)-based piezoelectric ultrasonic transducers, or the ultrasound sensor 20 may include capacitive micromachined ultrasound transducers (CMUT) in which the energy transduction is provided due to a change in capacitance.

The ultrasound sensor 20 may further include an ultrasound focusing lens, which may be positioned over the transducer array, and which may form a part of the sensing surface. The focusing lens may be any lens operable to focus a transmitted ultrasound beam from the transducer array toward a patient and/or to focus a reflected ultrasound beam from the patient to the transducer array. The ultrasound focusing lens may have a curved surface shape in some embodiments. The ultrasound focusing lens may have different shapes, depending on a desired application, e.g., a desired operating frequency, or the like. The ultrasound focusing lens may be formed of any suitable material, and in some embodiments, the ultrasound focusing lens is formed of a room-temperature-vulcanizing (RTV) rubber material.

In some embodiments, first and second membranes are positioned adjacent to opposite sides of the ultrasound sensor 20 and form a part of the sensing surface. The membranes may be formed of any suitable material, and in some embodiments, the membranes are formed of a room-temperature-vulcanizing (RTV) rubber material. In some embodiments, the membranes are formed of a same material as the ultrasound focusing lens.

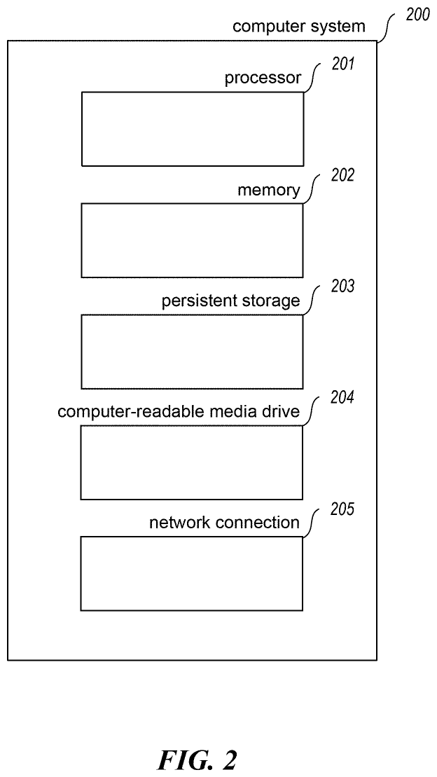

FIG. 2 is a block diagram showing some of the components typically incorporated in at least some of the computer systems and other devices on which the facility operates. In various embodiments, these computer systems and other devices 200 can include server computer systems, cloud computing platforms or virtual machines in other configurations, desktop computer systems, laptop computer systems, netbooks, mobile phones, personal digital assistants, televisions, cameras, automobile computers, electronic media players, physiological sensing devices, and/or their associated display devices, etc. In various embodiments, the computer systems and devices include zero or more of each of the following: a processor 201 for executing computer programs and/or training or applying machine learning models, such as a CPU, GPU, TPU, NNP, FPGA, or ASIC; a computer memory 202 for storing programs and data while they are being used, including the facility and associated data, an operating system including a kernel, and device drivers; a persistent storage device 203, such as a hard drive or flash drive for persistently storing programs and data; a computer-readable media drive 204, such as a floppy, CD-ROM, or DVD drive, for reading programs and data stored on a computer-readable medium; and a network connection 205 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components.

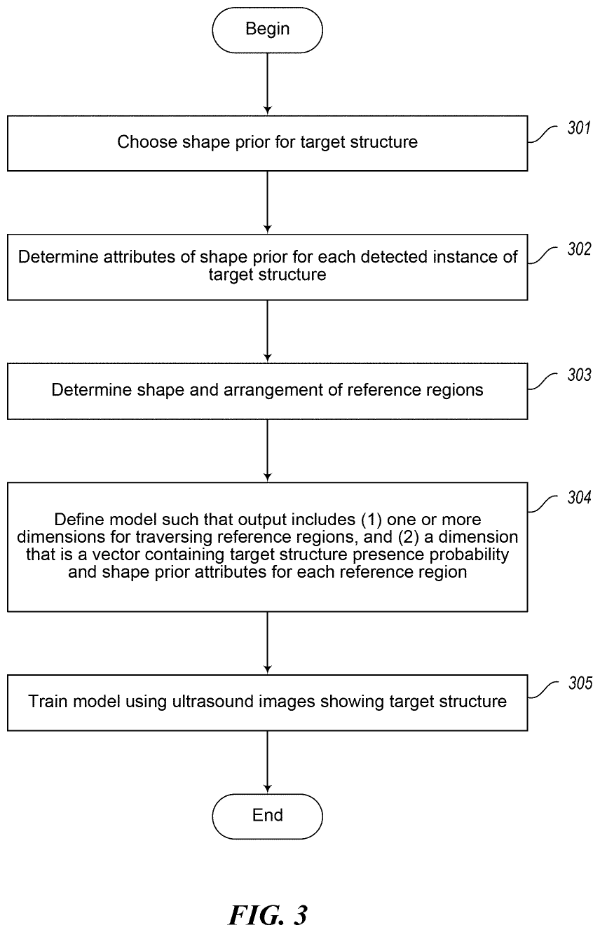

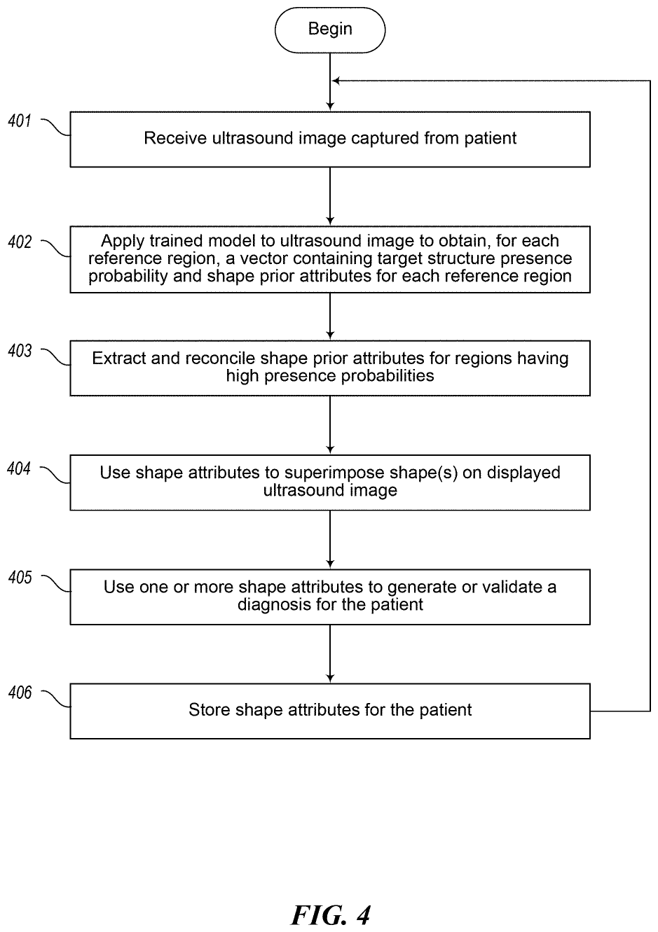

FIG. 3 is a flow diagram showing a process performed by the facility in some embodiments to establish and train a model for identifying a particular target anatomical structure. Details of applying this process and the one shown in FIG. 4 are discussed below with respect to a number of specific examples.

In act 301, the facility chooses a shape prior for the target structure. As discussed below in more detail, the facility typically chooses a simple shape that best matches the shape of common examples of the target structure in typical ultrasound images. For example, for generally round aortal cross-sections, the facility chooses an elliptical shape prior; for generally triangular or wedge-shaped pulmonary B-lines, the facility chooses a circle-sector shape prior.

In act 302, the facility determines attributes that can be used to fit the shape prior chosen in act 301 to each detected instance of the target structure. In the case of an elliptical shape prior chosen for aortal cross-sections, in some embodiments, the facility determines the following attributes: X and Y coordinates of the center of the ellipse; long-axis and short-axis diameters of the ellipse; and angle of rotation between the long-axis of the ellipse in the center of the ultrasound cone. In some embodiments, these attributes also include a presence probability attribute to represent the likelihood that each reference region contains an instance of the target structure.

In act 303, the facility determines the shape and arrangement of reference regions. In the case of aortal cross-sections, in some embodiments, the facility determines that a grid of rectangular reference regions will be used. In the case of pulmonary B-lines, the facility determines that a series of circle-sectors will be used.

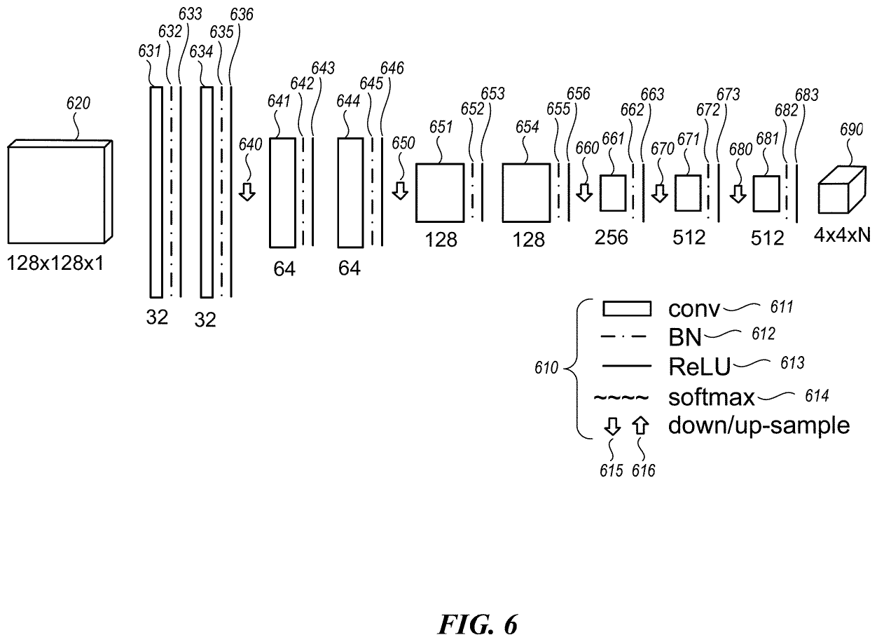

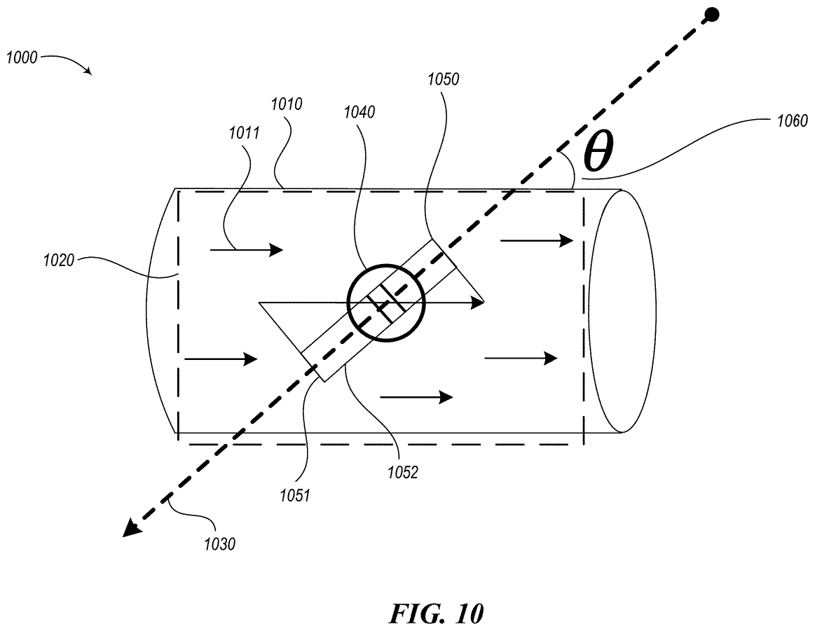

In act 304, the facility defines a model whose input is an ultrasound image, and whose output is a multidimensional tensor. The output tensor includes one or more dimensions for traversing the reference regions, and an additional dimension that is a vector containing (a) a target structure presence probability for the reference region, and (b) attribute values fitting the shape prior to a target structure instance occurring in the reference region. Example model definitions are discussed below in connection with FIGS. 6 and 10.

In act 305, the facility trains the model defined in act 304 using ultrasound images showing the target structure. After act 305, this process concludes.

FIG. 4 is a flow diagram showing a process performed by the facility in some embodiments to detect instances of a target structure visualized in an ultrasound image. In act 401, the facility receives an ultrasound image captured from a person, such as a patient. In act 402, the facility applies the model trained by the facility to the ultrasound image received in act 401 to obtain an output tensor. For each reference region, the output tensor contains a vector containing (a) a target structure presence probability for the reference region, and (b) attribute values fitting the shape prior to a target structure instance occurring in the reference region. In act 403, the facility extracts and reconciles the attribute values from reference regions having high presence probabilities. In some embodiments, the facility uses a configurable threshold value to determine which reference regions' presence probabilities are high enough. In some embodiments, the facility sets this configurable threshold value as part of validation performed on the model using additional training images. In act 404, the facility uses the shape prior attributes determined in act 403 to superimpose each fitted shape over a display of the ultrasound image, highlighting the target structure instances detected in the ultrasound image. In act 405, the facility uses one or more of the shape attributes determined in act 403 to generate or validate a diagnosis for the patient. In act 406, the facility stores the shape attributes determined in act 403 for the patient, such as in connection or association with the ultrasound image. After act 406, the facility continues in act 401 to receive the next ultrasound image, either from the same patient or a different patient.

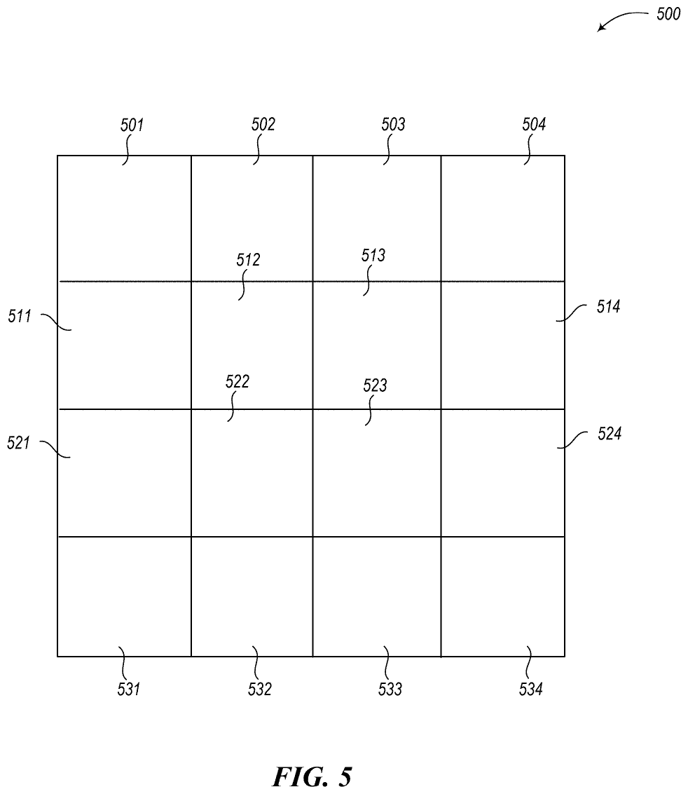

FIGS. 5-7 relate to detecting aortal cross-sections in ultrasound images of the heart. In some embodiments, the facility selects an ellipse as the shape prior to use for this application. In some embodiments, the facility selects a grid of rectangular reference regions as part of defining the model for this application.

FIG. 5 is a reference region layout diagram showing a grid of rectangular reference regions used by the facility in some embodiments. The diagram 500 includes rows of rectangular reference regions, such as a first row of reference regions 501-504, second row of reference regions 511-514, a third row of reference regions 521-524, and a fourth row of reference regions 531-534. In various embodiments, the reference regions are square or non-square rectangles. In various embodiments, the facility uses a two-dimensional array of reference regions having a variety of numbers of reference regions, of a variety of sizes. In some embodiments, reference regions that do not intersect with the ultrasound cone of ultrasound images are omitted or ignored.

FIG. 6 is a model architecture diagram showing a model architecture used by facility in some embodiments to accommodate a grid of rectangle reference regions. The model 600 is shown with respect to a key 610. The key shows symbols used in the diagram to represent 2D Convolutional layers 611, 2D Batch normalization layer 612, Leaky ReLU activation function layers 613, softmax layers 614, down-sample layers 615, and up-sample layers 616.

The model takes a 128×128×1 ultrasound image 620 as its input, and produces a 4×4×N tensor 690 as its output. The model first subjects the input ultrasound image to a convolutional block made up of 2D convolutional layer 631, 2D batch normalization layer 632, and leaky relu activation function layer 633. The model then proceeds to a convolutional block made up of 2D convolutional layer 634, 2D batch normalization layer 635, and leaky relu activation function layer 636. The model then proceeds to a downsample layer 640. The model then proceeds to a convolutional block made up of 2D convolutional layer 641, 2D batch normalization layer 642, and leaky relu activation function layer 643. The model then proceeds to a convolutional block made up of 2D convolutional layer 644, 2D batch normalization layer 645, and leaky relu activation function layer 646. The model then proceeds to a downsample layer 650. The model then proceeds to a convolutional block made up of 2D convolutional layer 651, 2D batch normalization layer 652, and leaky relu activation function layer 653. The model then proceeds to a convolutional block made up of 2D convolutional layer 654, 2D batch normalization layer 655, and leaky relu activation function layer 656. The model then proceeds to a downsample layer 660. The model then proceeds to a convolutional block made up of 2D convolutional layer 661, 2D batch normalization layer 662, and leaky relu activation function layer 663. The model then proceeds to a downsample layer 670. The model then proceeds to a convolutional block made up of 2D convolutional layer 671, 2D batch normalization layer 672, and leaky relu activation function layer 673. The model then proceeds to a downsample layer 680. The model then proceeds to a convolutional block made up of 2D convolutional layer 681, 2D batch normalization layer 682, and leaky relu activation function layer 683. Leaky relu activation function layer 683 produces the output tensor. In various embodiments, the facility uses a variety of neural network architectures and other machine learning model architectures to produce similar results.

In some embodiments, the facility allocates the first two dimensions of the output tensor to identify each of the reference regions, such as in a grid of 4×4 reference regions. In some embodiments, the facility selects the following ellipse shape prior attributes for use in the model it defines for detecting aortal cross-sections: probability of presence, center X and Y coordinates, long-axis diameter and rotational angle, and short-axis diameter. Thus, the N value sizing the vector that makes up the final dimension of the output tensor for the aortal cross-section application is 6.

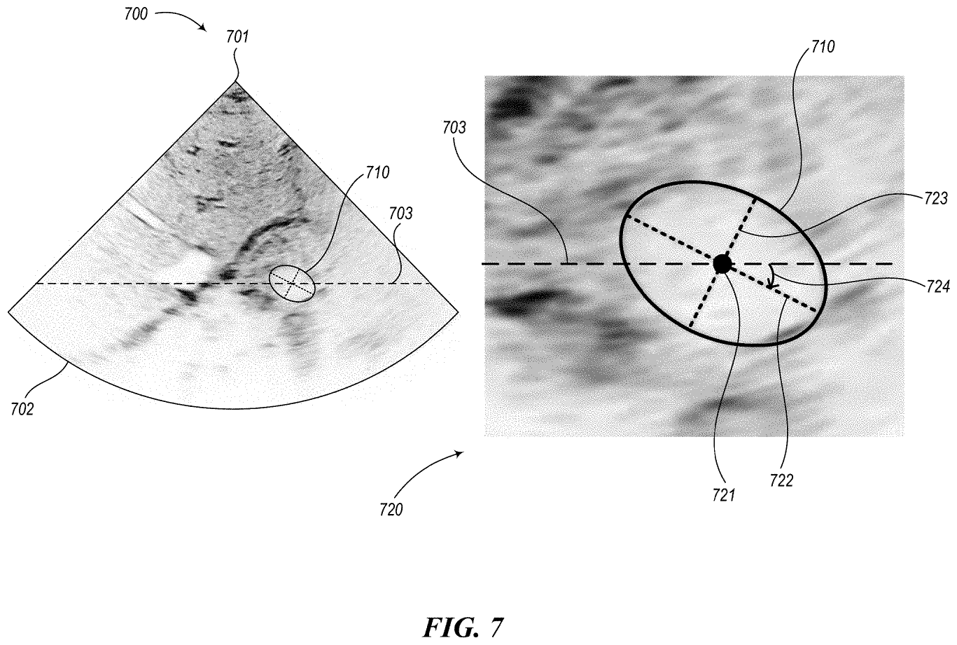

FIG. 7 is an ultrasound drawing of an ultrasound image showing an aortal cross-section detected by the facility. The ultrasound image 700 is shaped like a circle-sector—sometimes called a “cone”—with a top and 701 nearest the probe, and a bottom end 702 furthest from the probe. This ultrasound image and those discussed below have been black/white inverted to make them more reproducible and intelligible. The drawing shows the ellipse shape prior 710 fitted to the detected aortal cross-section by the facility. Also shown is a horizontal line 703 through the center of the ellipse. The attribute values determined by the facility to fit this ellipse to the aortal cross-section are as follows: X and Y coordinates of a center point 721 of the ellipse; the diameter 722 along the long axis of the ellipse; the diameter 723 along the short axis of the ellipse; and a directed angle 724 from the horizontal line through the center of the ellipse to the long axis of the ellipse. In some embodiments, the facility uses the long-axis diameter to identify potential aneurysm sites.

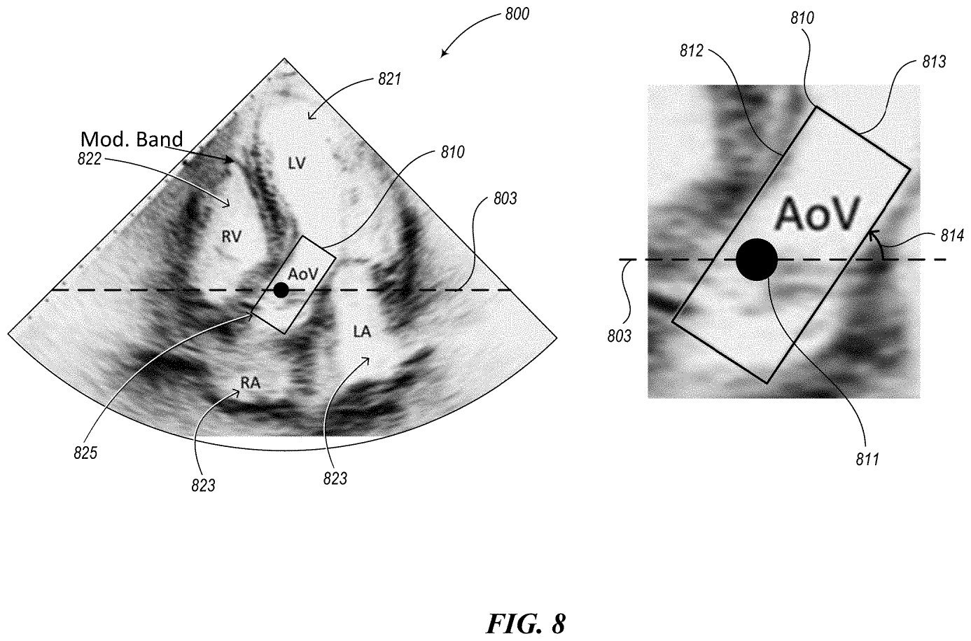

FIG. 8 relates to detecting LVOTs in ultrasound images of the heart. In some embodiments, the facility selects a rectangle as the shape prior to use for this application. In some embodiments, the facility selects a grid of rectangular reference regions as part of defining the model for this application. In some embodiments, the facility defines the model for this application based on the one shown in FIG. 6 and discussed above. In some embodiments, the facility selects the following rectangle shape prior attributes for use in the model it defines for detecting LVOTs: probability of presence, center X and Y coordinates, length, width, and long-axis rotational angle. Thus, the N value sizing the vector that makes up the final dimension of the output tensor for the LVOT application is 6.

FIG. 8 is an ultrasound drawing of an ultrasound image showing an LVOT detected by the facility. The ultrasound image 800—an apical five-chamber view of the heart—shows structures including left ventricle 821, right ventricle 822, right atrium 823, left atrium 824, and aortic valve 825. The image also shows the rectangular shape prior 810 fitted to the LVOT, and a horizontal line 803 through the center of the rectangle. The attribute values determined by the facility to fit this rectangle to the LVOT are as follows: X and Y coordinates of a center point 811; length 812; width 813; and a directed angle 814 from the horizontal line through the center of the rectangle to the long side of the rectangle. In some embodiments, the facility uses the location, size, and orientation of the LVOT to place a Doppler sample gate to determine an LVOT velocity-time integral for evaluating cardiac systole function.

In some embodiments, the facility assists the operator in aligning the rotational angle of the scanning axis to be parallel to the fitted rectangle. In some such embodiments, the facility determines (1) the orientation of the LVOT rectangle, and (2) the directed angle from the scanning axis to a line between the origin of the ultrasound cone and the center of the LVOT rectangle. If the LVOT orientation is aligned with the scanning axis, the facility indicates to the user that the orientation is optimal for Doppler data acquisition. If the LVOT orientation is not parallel to the scanning axis, the facility indicates to the user that the probe needs to be angled left or right (depending on the how the LVOT orientation and the scanning axis align). In the example shown in FIG. 8, the facility indicates to the user that the probe should be angled to the left side to align LVOT parallel to the scanning axis.

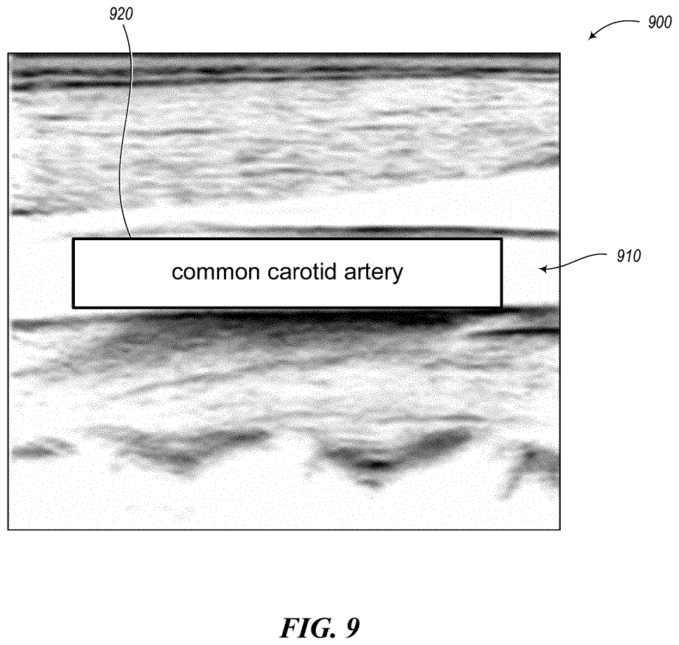

FIGS. 9-10 relate to detecting the interior of a blood vessel, such as for the placement of a vascular Doppler gate to determine blood velocity through the blood vessel. In some embodiments, the facility fits a rotated rectangle shape prior to the interior of the blood vessel, using a rectangular reference grid. To do so, in some embodiments, the facility selects the following rectangle shape prior attributes for use in the model it defines for detecting blood vessel interiors: probability of presence, center X and Y coordinates, length, width, and long-axis rotational angle. Thus, the N value sizing the vector that makes up the final dimension of the output tensor for the blood vessel interiors application is 6.

FIG. 9 is an ultrasound drawing of an ultrasound image showing a blood vessel interior detected by the facility. The drawing 900 shows only a rectangular portion of the ultrasound cone. The drawing shows a rotatable rectangular shape prior 920 fitted to the interior 910 of a blood vessel, here the common carotid artery. In some embodiments, the facility uses this fitted rectangle to establish a gate for performing vascular Doppler analysis. In some embodiments, in a manner similar to the LVOT application discussed above, the facility uses the rotational angle of the fitted rectangle to assess the blood vessel's alignment with the scanning axis, and directs the operator to move the transducer to better align them.

In some vascular Doppler cases, the operator will not be able to orient the probe parallel to the blood flow due to anatomical constraints. However, because of the tube shape of the vasculature, in some embodiments the facility assumes homogeneous direction of the blood flow and applies Doppler angle correction to adjust the calculation of blood velocity. In particular, band on the angle between the scanning axis and the vasculature θ, in some embodiments the facility adjusts the blood velocity using the following equation:

v_{correct}=v_{Doppler}/cos(θ)

where v_{Doppler} is the measured Doppler velocity. To improve system reliability, in some embodiments the facility applies such correction only when θ is smaller than a certain threshold, such as 60 degrees.

FIG. 10 is a conceptual diagram showing aspects of the facility's estimation of blood velocity in the direction of the blood vessel's length. The drawing 1000 shows the blood vessel 1010 as a cylinder. Blood is flowing along the length of the blood vessel as shown by directional arrow 1011. The facility fits rectangle 1020 to the interior of the blood vessel. A Doppler gate 1044 measuring blood flow velocity has been placed along a line 1030 from the ultrasound origin—i.e., the top of the ultrasound cone—through the center of the fitted rectangle, at the point of the center of the fitted rectangle. In particular, the Doppler gate placed in this way measures the velocity of blood in the direction of line 1030, as opposed to in the direction 1011 of blood flow along the blood vessel's length as desired. The facility thus uses angle θ 1060 in the manner discussed above to adjust the Doppler blood velocity results to reflect velocity in the direction of blood flow along the blood vessel's length.

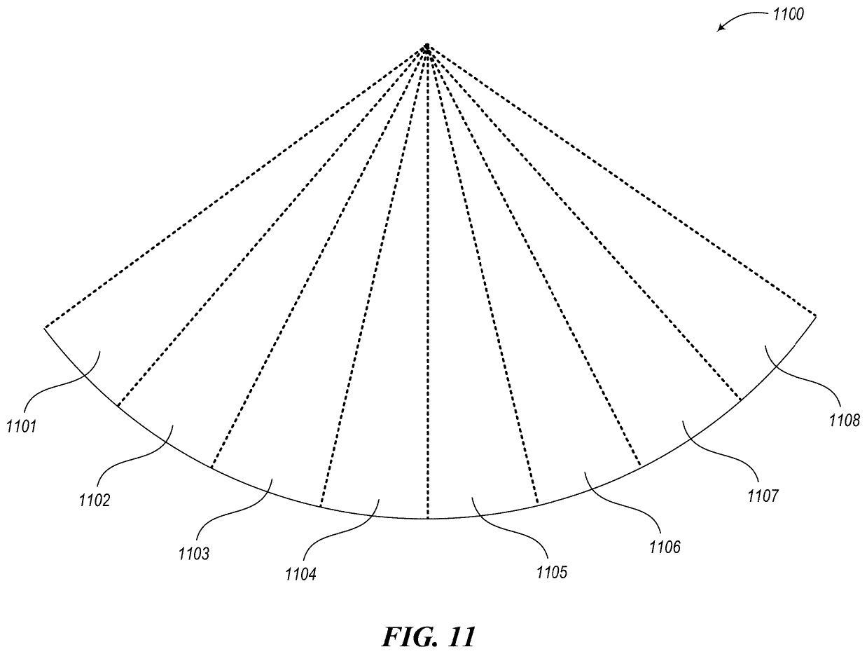

FIGS. 11-15 relate to detecting pulmonary B-Lines in ultrasound images of the lung. In some embodiments, the facility selects a circle-sector as the shape prior to use for this application. In some embodiments, the facility selects a series of top-to-bottom circle-sector reference regions as part of defining the model for this application.

FIG. 11 is a reference region layout diagram showing a series of top-to-bottom circle-sector reference regions used by the facility in some embodiments. The diagram 1100 includes circle-sector reference regions 1101-1108. In various embodiments, the circle-sector reference regions have uniform angular width, or variable angular width, such as being narrower toward the center of the ultrasound image.

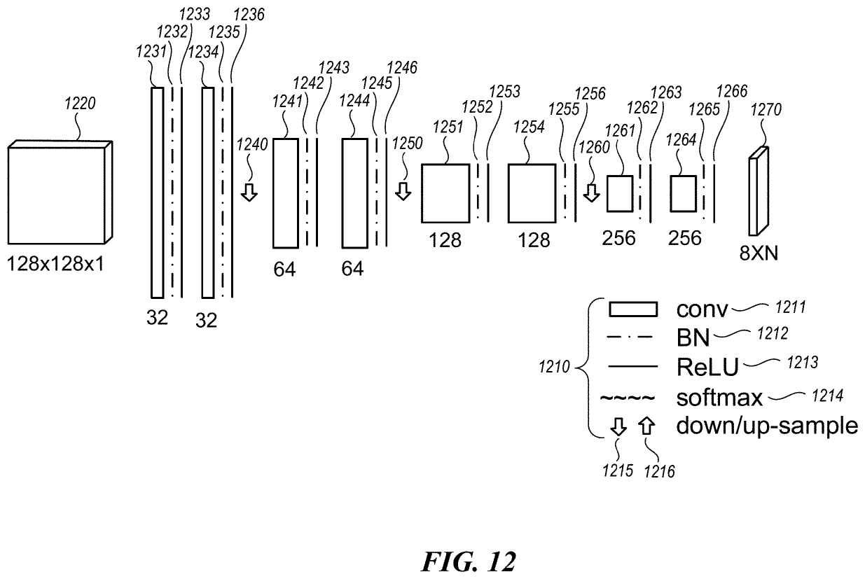

FIG. 12 is a model architecture diagram showing a model architecture used by facility in some embodiments to accommodate a series of circle-sector reference regions. The model 1000 1200 is similar to model 600 shown in FIG. 6, subjecting an ultrasound image 1220 to network layers 1231-1266, similar network layers 631-666, to produce an output tensor 1270. The output tensor 1270 has a single dimension to identify each of the reference regions, such as a series of 8 reference regions. In some embodiments, the facility selects the following circle-sector shape prior attributes for use in the model it defines for detecting B-lines: probability of presence, directed angle between the height of the ultrasound image and the center of the sector, and angular width of the sector. Thus, the N value sizing the vector that makes up the final dimension of the output tensor for the B-line application is 3.

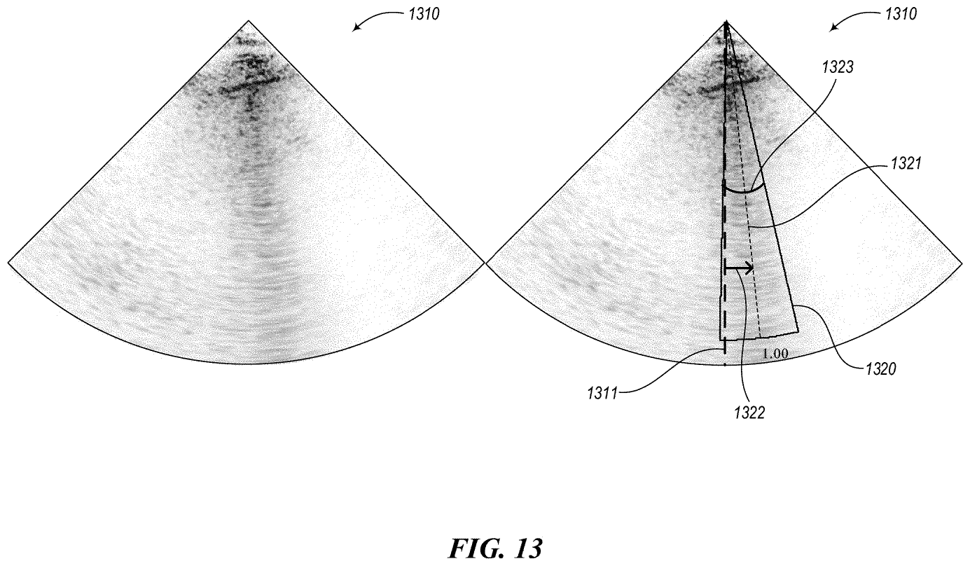

FIGS. 13-15 are ultrasound drawing of ultrasound images showing B-lines detected by the facility. FIG. 13 is a first ultrasound drawing of ultrasound images showing B-lines detected by the facility. The ultrasound image 1310 shows a circle-sector shape prior 1320 fitted to a single detected B-line. The attribute values determined by the facility to fit circle-sector to the B-line are as follows: directed angle 1322 between the height 1311 of the ultrasound image and the center of the sector 1321, and angular width of the sector 1323. In some embodiments, the facility uses the number, angular location, and/or angular width of B-lines to diagnose and/or grade pulmonary edema.

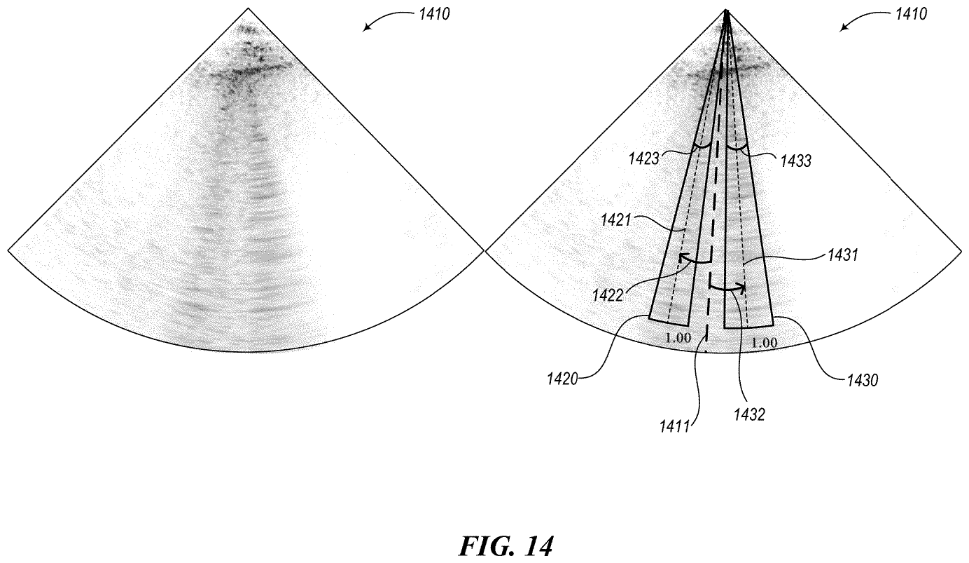

FIG. 14 is a second ultrasound drawing of ultrasound images showing B-lines detected by the facility. The ultrasound image 1410 shows circle-sector shapes prior 1420 and 1430 fitted to two detected B-lines. The attribute values determined by the facility to fit circle-sector 1420 to the first B-line are as follows: directed angle 1422 between the height 1411 of the ultrasound image and the center of the sector 1421, and angular width of the sector 1423. The attribute values determined by the facility to fit circle-sector 1430 to the second B-line are as follows: directed angle 1432 between the height 1411 of the ultrasound image and the center of the sector 1431, and angular width of the sector 1433.

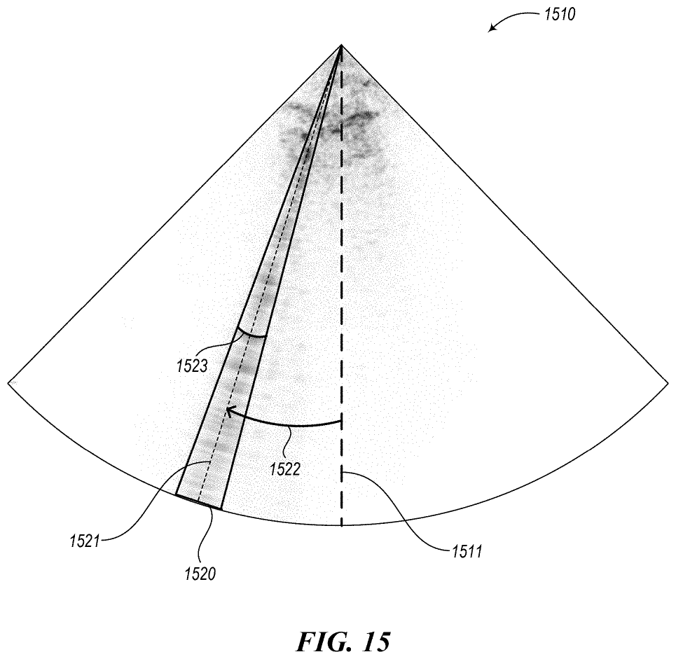

FIG. 15 is a third ultrasound drawing of ultrasound images showing B-lines detected by the facility. The ultrasound image 1510 shows a circle-sector shape prior 1520 fitted to a single detected B-line. The attribute values determined by the facility to fit circle-sector to the B-line are as follows: directed angle 1522 between the height 1511 of the ultrasound image and the center of the sector 1521, and angular width of the sector 1523.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A system, comprising:
an ultrasound transducer; and
a computing device, the computing device comprising:
a communication interface configured to directly receive ultrasound echo data sensed by the ultrasound transducer from a person, the received ultrasound echo data comprising a sequence of ultrasound images;
a memory configured to:
store a trained machine learning model for producing inferences each in response to an ultrasound image in the sequence;
a processor configured to:
for each ultrasound image of the sequence, in response to its receipt by the communications interface:
subject the ultrasound image to the machine learning model to produce an inference, the inference (1) identifying one or more occurrences of a distinguished anatomical structure occurring in the ultrasound image; and (2) for each identified occurrence, specifying attribute values fitting to the identified occurrence a distinguished shape selected to correspond to typical visualizations of the distinguished anatomical structure; and
store the ultrasound image and the specified attribute values in a manner that associates the stored ultrasound image and specified attribute values.

2. The system of claim 1, the processor further being configured to train the stored machine learning model based on ultrasound images in which the distinguished anatomical structure is visualized.

3. The system of claim 1, the processor further being configured to display, superimposed upon the ultrasound image, the distinguished shape fitted to the identified occurrences of the distinguished anatomical structure in accordance with the stored attribute values.

4. The system of claim 1, the processor further being configured to determine a diagnosis on the basis of at least one of the specified attribute values.

5. The system of claim 1, the processor further being configured to determine a treatment for the person on the basis of at least one of the specified attribute values.

6. The system of claim 1 wherein a first one of the specified attribute values reflects an angle of rotation of the fitted distinguished shape relative to the active surface of the transducer.

7. The system of claim 6, the processor further being configured to:
based upon the angle of rotation of the fitted distinguished shape, cause a direction to be conveyed to an operator to reposition the transducer relative to the person.

8. The system of claim 6 wherein the distinguished anatomical structure is the left ventricle outflow track,
and wherein the distinguished shape is a rectangle,
and wherein the specified attribute values further comprise second attribute values reflecting length, width, and location of the rectangle fitted to the identified occurrence of the left ventricle outflow track.

9. The system of claim 1, the processor further being configured to:
for a distinguished image of the sequence:
use the fitted distinguished shape to place a Doppler gate in the image; and
initiate Doppler analysis with respect to the placed Doppler gate.

10. The system of claim 9 wherein a first one of the specified attribute values reflects an angle of rotation of the fitted distinguished shape relative to the active surface of the transducer,
the processor further being configured to:
receive a result of the initiated Doppler analysis; and
use the value of the first attribute to adjust the received result to correct for the reflected angle of rotation.

11. The system of claim 10, the processor further being configured to:
cause the adjusted result to be displayed.

12. The system of claim 10, the processor further being configured to:
cause the adjusted result to be persistently stored for the person.

13. The system of claim 6 wherein the distinguished anatomical structure is
the interior of a blood vessel,
and wherein the distinguished shape is a rectangle,
and wherein the specified attribute values further comprise second attribute values reflecting length, width, and location of the rectangle fitted to the identified occurrence of the blood vessel interior.

14. The system of claim 1 wherein the distinguished shape is nonrectangular.

15. The system of claim 14 wherein the distinguished shape is an ellipse.

16. The system of claim 15 wherein the distinguished anatomical structure is an aortal cross-section.

17. The system of claim 14 wherein the distinguished shape is a circle-sector.

18. The system of claim 17 wherein the distinguished anatomical structure is an aortal cross-section.

19. The system of claim 1 wherein the machine learning model is configured to analyze an ultrasound image with respect to an arrangement reference regions within the ultrasound image having a selected non-rectangular shape.

20. The system of claim 1 wherein the machine learning model is configured to analyze an ultrasound image on the basis of an arrangement reference regions within the ultrasound image having a circle-sector shape.

21. One or more computer memories collectively storing a data structure, the data structure comprising:
data comprising a trained state of a neural network configured to predict parameter values fitting a distinguished shape to an occurrence of a distinguished anatomical feature in an ultrasound image,
such that the contents of the model are usable to apply the trained neural network to an ultrasound image to predict the parameter values that fit the distinguished shape to an occurrence of the distinguished anatomical feature.

22. The one or more computer memories of claim 21 wherein the predicted parameter values comprise a first parameter value specifying an angle of rotation of the distinguished shape relative to the two dimensions of the ultrasound image.

23. The one or more computer memories of claim 22 wherein the distinguished anatomical feature is the left ventricle outflow track,
and wherein the distinguished shape is a rectangle,
and wherein the predicted parameter values further comprise second parameter values reflecting length, width, and location of the rectangle fitted to the identified occurrence of the left ventricle outflow track.

24. The one or more computer memories of claim 22 wherein the distinguished anatomical structure is the interior of a blood vessel,
and wherein the distinguished shape is a rectangle,
and wherein the specified attribute values further comprise second attribute values reflecting length, width, and location of the rectangle fitted to the identified occurrence of the blood vessel interior.

25. The one or more computer memories of claim 21 wherein the distinguished shape is non-rectangular.

26. The one or more computer memories of claim 25 wherein the distinguished shape is an ellipse.

27. The one or more computer memories of claim 26 wherein the distinguished anatomical feature is an aortal cross-section.

28. The one or more computer memories of claim 25 wherein the distinguished shape is a circle-sector.

29. The one or more computer memories of claim 28 wherein the distinguished anatomical feature is a pulmonary B-line.

30. The one or more computer memories of claim 21 wherein the neural network is configured to analyze an ultrasound image with respect to an arrangement reference regions within the ultrasound image having a selected shape.

31. The one or more computer memories of claim 30 wherein the selected shape is non-rectangular.

32. The one or more computer memories of claim 31 wherein the selected shape is a circle-sector.

33. A method in a computing system, comprising:
receiving an ultrasound image;
subjecting the ultrasound image to a detection model to obtain, for each of one or more occurrences of a target structure appearing in the ultrasound image, a set of parameter values fitting a distinguished shape to the target structure occurrence; and
storing the obtained one or more parameter value sets in connection with the ultrasound image.

34. The method of claim 33, further comprising training the model using training ultrasound images in which the target structure appears.

35. The method of claim 33, further comprising:
causing the ultrasound image to be displayed; and
augmenting the displayed ultrasound images with the distinguished shape, fitted in accordance with each of the sets of parameter values.

36. The method of claim 33 wherein each set of parameter values comprises a first parameter value specifying an angle of rotation of the distinguished shape relative to the two dimensions of the ultrasound image.

37. The method of claim 33, further comprising:
based upon the angle of rotation of the fitted distinguished shape, causing a direction to be conveyed to an operator to reposition the transducer relative to the person.

38. The method of claim 36 wherein the target structure is the left ventricle outflow track,
and wherein the distinguished shape is a rectangle,
and wherein each set of parameter values further comprises second parameter values reflecting length, width, and location of the rectangle fitted to the identified occurrence of the left ventricle outflow track.

39. The method of claim 36 wherein the distinguished anatomical structure is the interior of a blood vessel,
and wherein the distinguished shape is a rectangle,
and wherein the specified attribute values further comprise second attribute values reflecting length, width, and location of the rectangle fitted to the identified occurrence of the blood vessel interior.

40. The method of claim 33, further comprising:
for a distinguished image of the sequence:
using the fitted distinguished shape to place a Doppler gate in the ultrasound image; and
initiating Doppler analysis with respect to the placed Doppler gate.

41. The method of claim 40 wherein a first one of the obtained parameter values reflects an angle of rotation of the fitted distinguished shape relative to the active surface of a transducer used to capture the received ultrasound image, the method further comprising:
receiving a result of the initiated Doppler analysis; and
using the first parameter value to adjust the received result to correct for the reflected angle of rotation.

42. The method of claim 41, further comprising:
causing the adjusted result to be displayed.

43. The method of claim 41, further comprising:
causing the adjusted result to be persistently stored for the person.

44. The method of claim 33 wherein the distinguished shape is non-rectangular.

45. The method of claim 44 wherein the distinguished shape is an ellipse.

46. The method of claim 45 wherein the target structure is an aortal cross-section.

47. The method of claim 44 wherein the distinguished shape is a circle-sector.

48. The method of claim 47 wherein the target structure is a pulmonary B-line.
